(19) United States
(12) Patent Application Publication
(10) Pub. No.: US 2021/0356147 A1
(43) Pub. Date: Nov. 18, 2021

(54) Air Freshener Device

(71) Applicant: Robert Basile, Sunrise, FL (US)

(72) Inventor: Robert Basile, Sunrise, FL (US)

(21) Appl. No.: 17/120,469

(22) Filed: Dec. 14, 2020

Related U.S. Application Data
(60) Provisional application No. 63/023,649, filed on May 12, 2020.

(51) Int. Cl. F24F 3/16; F24F 13/08; F24F 11/00; F24F 7/06

(52) U.S. Cl. CPC F24F 3/16; F24F 13/082; F24F 2003/1689; F24F 7/065; F24F 11/0001

(57) ABSTRACT

An air freshening device for attachment to a forced air vent is disclosed herein. The air freshening device having a vented housing, a reservoir containing a dispersible fragrant material, and an fragrance adjusting mechanism for adjusting the amount of fragrant material dispersed into an air stream emitted from the forced air vent. In one embodiment, the fragrance adjusting mechanism is remotely controllable, and the amount of fragrant material dispersed into an air stream may also be controlled by the angular placement of the vented housing relative to the forced air vent.

### CROSS-REFERENCE TO RELATED APPLICATION

The present application claims priority to, and the benefit of, U.S. Provisional Application No. 63/023,649, which was filed on May 12, 2020 and is incorporated herein by reference in its entirety.

### BACKGROUND

The present invention relates generally to the field of air fresheners. More specifically, the present invention relates to an air freshening device that can easily be attached to residential or commercial heating, ventilation and air conditioning (HVAC) system vents to disperse fresh scents, and utilizes the forced air being pushed through the ventilation system to distribute the scent throughout the area. The air freshening device preferably comprises a clip designed to attach the air freshening device to the HVAC vent, a fragrant material, and a reservoir attached to the clip for holding the fragrant material, wherein the HVAC blower causes the fragrant material stored in the reservoir to be dispersed throughout the room via the forced air. Also, the air freshening device may further comprise an adjusting mechanism to enable the user to adjust the amount of fragrant material that is dispersed during any particular period of time. Accordingly, the present specification makes specific reference thereto. Nonetheless, it should be appreciated that aspects of the present invention are also equally amenable to other like applications, devices and methods of manufacture.

Keeping a room or other enclosed space clean and fresh smelling is a laborious and never-ending battle in many homes and offices. Stale odors, poor circulation, tobacco smoke, food odors, pet odors, and the like all combine to offend the olfactory senses of anyone entering a room, and may cause embarrassment to the homeowner or other person responsible for maintaining the cleanliness of the space.

By way of background, most commonly used air freshening devices used in the home and other enclosed spaces consist of incense sticks, diffusers, scented candles, aerosol sprays, or aromatic balls, which are placed into an area where the air needs to be freshened. However, many of such devices take up floor space, have a limited range of effectiveness and/or detract from the aesthetics of the home. Additionally, many of said air freshening devices may be harmful to children and pets. For example, scented candles are a well-documented fire hazard, particularly when left unattended, and incense sticks and aroma balls may be both poisonous and/or a choking hazard to, for example, a dog or a young child. Moreover, many of such air freshening devices are typically single use devices that must be discarded once used or consumed, and it can be expensive to have to routinely replace the same with new products.

It has also been a common practice to use room air fresheners in the form of a sponge or matrix-like material containing a scented product or ingredient which is then released into the surrounding air as the product is suspended by a string, hook or the like from a support. However, such devices are not aesthetically pleasing, and can interfere with individuals moving around in the enclosed space (e.g., if suspended from a ceiling in the middle of the room). Still other air freshening devices are comprised of a housing that holds a volatile material in connection with an electrically heated vapor dispensing device. However, such air freshening devices require an electrical connection, either through a normal electrical supply or a battery, and can therefore be costly to operate and/or replace.

Additionally, a common problem with all of the above referenced air freshening devices is that the freshening smell is overpowering at first, and then the aroma quickly diminishes over time. Also, there is no mechanism to regulate the strength of the fragrance of the air freshening device to suit the needs and preferences of the user. Another common problem with traditional air fresheners is that the freshening aroma is not evenly distributed throughout the room or other enclosed space, and is only concentrated in the area in which it is located and/or released. More specifically, once the scent or fragrance is released, no means is provided for the distribution thereof. Stated differently, existing air freshening devices do not take advantage of the forced air or HVAC sources already servicing the room or enclosed space, and require the user to purchase a separate can, nozzle, or aerosol for distributing the fragrance, which can be expensive, time consuming, and requires disposal after use.

Therefore, there exists a long felt need in the art for an air freshening device that utilizes an existing HVAC ventilation system to force the scented air or fragrance into the room or other enclosed space in an efficient manner, and that evenly distributes freshening aroma throughout the entire room or enclosed space. There is a long felt need in the art for an air freshening device that allows a user to regulate the strength of the fragrance being emitted from the air freshener device to suit the needs and preferences of the user, and that easily and quickly attaches to an HVAC vent. Additionally, there is a long felt need in the art for an air freshening device that is refillable and that enables users to choose from different types of scents and fragrances. Moreover, there is a long felt need in the art for an air freshening device that is relatively safe for children, pets, and elderly individuals that may have a breathing condition, and that may further comprise anti-bacterial, anti-viral, anti-microbial and/or anti-fungal properties. Finally, there is a long felt need in the art for an air freshening device that is relatively inexpensive to manufacture, safe and easy to use, and that eliminates odors and distributes a pleasant fragrance into a room or other enclosed space with the assistance of forced air emanating from an existing HVAC system.

The subject matter disclosed and claimed herein, in one embodiment thereof, comprises an air freshening device to be used as an attachment to an existing HVAC vent. More specifically, the air freshening device comprises a clip for releasably attaching to, for example, a fin of an HVAC vent, a fragrance reservoir having a user selectable and refillable fragrance contained therein, and an adjusting mechanism for adjusting the amount of the strength of the fragrance being dispensed. The fragrance may be in the form of a gel, a mesh, an air freshener strip, a fabric or the like, and may be further comprised of one or more of an anti-bacterial, anti-viral, anti-microbial and/or anti-fungal solution or formulation.

In this manner, the air freshening device of the present invention accomplishes all of the forgoing objectives, and provides a relatively quick and easy solution to dispersing fresh scents and fragrances evenly into a room or other enclosed space according to the needs and/or preferences of the user. The improved air freshening device of the present invention is also simple in both design and use, economical, efficient in its construction and installation, and eliminates the need to purchase unnecessary and additional accessories to properly function.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, is an air freshening device comprising a clip designed to releasably attach the device to an HVAC vent, a supply or reservoir containing a fragrant material, and an adjusting mechanism to control the amount of fragrance dispersed from the device, wherein the fragrance of the device is released into the forced air that is expelled from the HVAC system through the vent, which in turn carries the fragrant material for distribution throughout the entire room or enclosed space. The fragrant material of the air freshening device may be in the form of a mesh, porous strips, a gel, a solid, a liquid, or the like.

The subject matter disclosed and claimed herein, in an alternative embodiment thereof, comprises a home air freshener system used in combination with a forced air system, such as an HVAC unit. More specifically, the home air freshener system if comprised of an air freshener device removably attached to an output of the HVAC unit so that the forced air emanating therefrom expels a fragrance throughout an entire room or other enclosed space. The fragrant material may be in the form of a mesh, porous strips, a gel, a solid, a liquid, or the like, and is housed in a reservoir of the air freshener device. The air freshener system may further comprise an adjustment means to control the amount of fragrant material being dispersed, and the fragrance may further comprise at least one of an anti-bacterial, anti-viral, anti-microbial and/or anti-fungal solution or formulation.

In a further embodiment of the present invention, a removable or clip-on type air freshener device for use with an HVAC vent is disclosed and includes a supply or reservoir for holding a fragrant material, an adjusting mechanism to adjust and control exposure and strength of the fragrance by regulating the rate of its evaporation into a stream of forced air generated by an HVAC, wherein the adjusting mechanism may be controlled remotely.

In still a further embodiment of the present invention, an air scenting device for use with an airflow vent is disclosed. The device includes a supply or reservoir to store a scent that does not require user activation, an attachment device for attaching the reservoir to the airflow vent, and a means for adjusting the amount of scent to be released from the scenting device throughout a room as per the preferences of the user.

In yet a further embodiment of the present invention, a method of introducing a fragrance into an environment comprising the steps of initially obtaining an air freshening device, and then removably attaching the air freshening device to the grill or output vent of a forced air heating or air conditioning system via a clip or other suitable attachment means. Next, the device is placed such that the air from the vent contacts the fragrant material present in a supply or reservoir of the device, which slowly diffuses the material into the air and dissolves or dissipates the supply. The strength of the release of fragrant material can be controlled by the user via an adjustment means, either manually or remotely.

In yet a further embodiment, an air freshening system is described and includes a forced air flow system that is provided in a room. The forced air flow system has at least one vent or register having at least one grill, and an air freshening device, wherein the device is comprised of a vented housing, a supply of scented materials stored therein, and a clip for attaching the air freshening device to the at least one grill. The supply of scented materials is phthalate-free, paraben free and International Fragrance Association (IFRA) compliant. The forced air flow system produces an air flow of between 400 and 1,000 CFM.

In a still further embodiment of the present invention, a scent diffusing device, is described and includes a container that is attached to a grill of a register or vent of an air flow system. The container has a housing with vents disposed on each of a front and rear surface. The container has a supply of scented material, with the scented material being phthalate-free, paraben free and International Fragrance Association (IFRA) compliant. The container further includes a scent adjusting device to control the diffusing of the scented material into a room from at least a low to a high setting. The container is attached to the grill or register by a replaceable clip, with the replaceable clip being able to provide an angular displacement of the container proximate to a face of the register or grill and air flow from the air flow system.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1a illustrates a perspective view of one possible embodiment of an air freshening device of the present invention in accordance with the disclosed architecture, wherein the air freshening device is comprised of an adjustment means;

FIG. 1b illustrates a close-up perspective view of one possible embodiment of the adjustment means of FIG. 1a in accordance with the disclosed architecture;

FIG. 2 illustrates a rear perspective view of one possible embodiment of an air freshening device of the present invention in accordance with the disclosed architecture, and having an attachment means in the form of a clip;

FIG. 3 illustrates a perspective view of one possible embodiment of an air freshening device of the present invention in accordance with the disclosed architecture, wherein the air freshening device is being attached to a forced air vent.

FIG. 4 illustrates a perspective view of one possible embodiment of an air freshening device of the present invention in accordance with the disclosed architecture, wherein the air freshening device is attached to a forced air vent and dispersing a fragrance into an enclosed space.

### DETAILED DESCRIPTION

The innovation is now described with reference to the drawings, wherein like referenced numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

As noted above, there is a long felt need in the art for an air freshening device that utilizes an existing forced air system, such as an HVAC ventilation system, to efficiently and thoroughly disperse a scented fragrance into an enclosed space, such as a room. There is also a long felt need in the art for an air freshening device that allows a user to regulate the amount of the fragrance being emitted from the device to suit the needs and preferences of the user, and that is removably attachable to a forced air vent. Additionally, there is a long felt need in the art for an air freshening device that is self-activating, refillable with various different fragrances, and that may further comprise at least one of an anti-bacterial, anti-viral, anti-microbial and/or anti-fungal solution. Finally, there is a long felt need in the art for an air freshening device that is relatively inexpensive to manufacture, safe and easy to use, and that eliminates odors and distributes pleasant fragrances into a room or other enclosed space with the assistance of forced air emanating from an existing HVAC system.

The innovative product of the present invention features an air freshening device for attachment to an HVAC vent and that comprises a clip, a supply or reservoir for containing and dispensing a fragrant material, a fragrant material stored in the reservoir and an adjusting mechanism. The adjusting mechanism allows the user to control the amount of fragrance that is released from the device into the forced air being expelled from the HVAC vent. The forced air, in turn, carries the fragrant material for distribution throughout an entire room or enclosed area to be treated. The fragrant material may be stored in the reservoir in many forms including, without limitation, a mesh, strips, a gel, a solid, a liquid or the like, and may further comprise at least one of an anti-bacterial, anti-viral, anti-microbial and/or anti-fungal solution or formulation for al so killing any germs, bacteria, viruses, or airborne pathogens.

Therefore, a novel air freshening device is disclosed having a user selectable and controllably released fragrant or scented material that is stored in a reservoir or supply chamber of the device and introduced into a forced air stream for distribution throughout an entire room. By way of background, the normal air flow for an HVAC unit in an average sized room ranges from 400 to 2,000 cubic feet per minute (CFM) depending on the location of the measurement, and the dimensions of the associated duct work. A standard or desirable amount of air flow for an enclosed space is roughly 1 cubic foot per minute per the square footage of the area to be heated or cooled. The present invention is designed to work with an HVAC unit having an airflow circulation of between 400 and 1,000 CFM, and more preferably between 400 and 800 CFM, though the same should not necessarily be construed as a limitation on the usefulness of the air freshening device of the present invention.

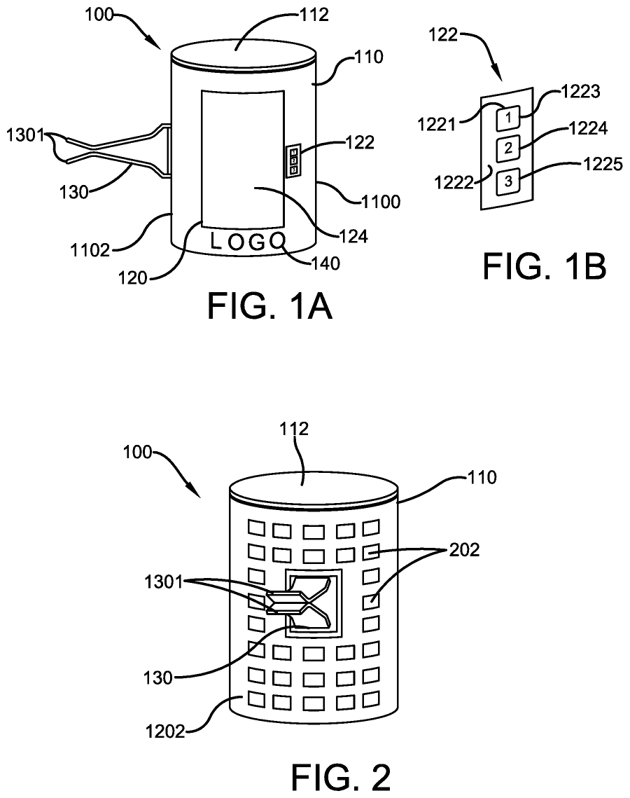

Referring initially to the drawings, FIG. 1a illustrates a perspective view of one possible embodiment of an air freshening device 100 of the present invention in accordance with the disclosed structure. The air freshening device 100 is used for dispensing various user selectable and controllable fragrances or scents through the forced air that emanates from an HVAC device, or other central heating and cooling system, in a consistent and uniform manner throughout a room or other enclosed apace. When referring to HVAC systems herein, it should be noted that the air freshening device 100 is also suitable for use with personal air conditioning and heating units, such as window units and portable units which may be moved from room to room.

As best shown in FIG. 1a, the air freshening device 100 is comprised of a housing 110 which, in one embodiment, is a generally cylindrical shaped structure having a reservoir or supply 120 filled with a fragrant material 124. Notwithstanding, the housing 110 may be in the form of other geometrical shapes, such as cubical, spherical, or any other geometric or non-geometric shape that satisfies the wants and needs of the user and that is readily attachable to an HVAC vent, as described more fully below. More specifically, the housing 110 comprises a front end 1100, a rear end 1102 and a lid 112, wherein the front end 1100 of the housing 110 may further comprise a plurality of openings or vents for releasing the fragrance or scent of the fragrant material 124 stored inside the reservoir 120 into the surroundings. The lid 112 is preferably hinged to rear end 1102 and repositionable between an open and a closed position. When opened, the lid 112 provides a user with a access to the reservoir 120, for example, to fill or refill the same with the fragrant material 124. As discussed more fully below, the fragrance or scented material 124 is preferably dispersed in an amount ranging from about 1 to 5 ppm, and having a particle size ranging from around 0.1 to 25 microns, depending on the setting of the adjustment mechanism 122.

As best shown in FIGS. 1a and 1b, the housing 110 is further comprised of a fragrance strength adjusting mechanism 122 that allows a user to manipulate or adjust the amount and/or strength of the fragrance or scent being released from the air freshening device 100 at any given time. The fragrance strength adjusting mechanism 122 may be a sliding button which the user slides over plate to adjust the strength of the fragrance as per the needs and requirements of the user, or a plurality of buttons. Alternatively, a knob may be present to partially close the openings/vents of the device 100 to regulate the fragrance released from the air freshening device 100, as described more fully below.

More specifically, FIG. 1b illustrates the fragrance scent strength adjusting mechanism 122 for the air freshening device 100 of the present invention in accordance with the disclosed architecture. In this particular embodiment, the fragrance strength adjusting mechanism 122 comprises a sliding button 1221 on a slidable plate 1222 that enables a user to adjust the strength or release condition of the fragrance or scent 124 to be diffused as per the preferences of the user. In a preferred embodiment, the slidable plate 1222 includes three modes of fragrance strength, namely a low strength mode 1223, a medium strength mode 1224, and a high strength mode 1225. When a user slides the button 1221 to the low strength mode 1223 setting on the slidable plate 1222, the air freshening device 100 operates at a relatively low strength and permits the device 100 to release a relatively low amount of fragrant material 124 from the device and into the forced air emanating from the HVAC unit. If the user slides the button 1221 to the medium strength mode setting 1224 on the slidable plate 1222, the air freshening device 100 operates at a medium strength and permits the device 100 to release a larger amount of fragrant material 124 from the air freshening device 100, as compared to that which is released in the low strength mode 1223. Likewise, when the user slides the button 1221 to the high strength mode setting 1222 on the slidable plate 1222, the air freshening device 100 operates at a relatively high strength and permits the device 100 to release more of fragrant material 124 into the forced air stream than with any other setting.

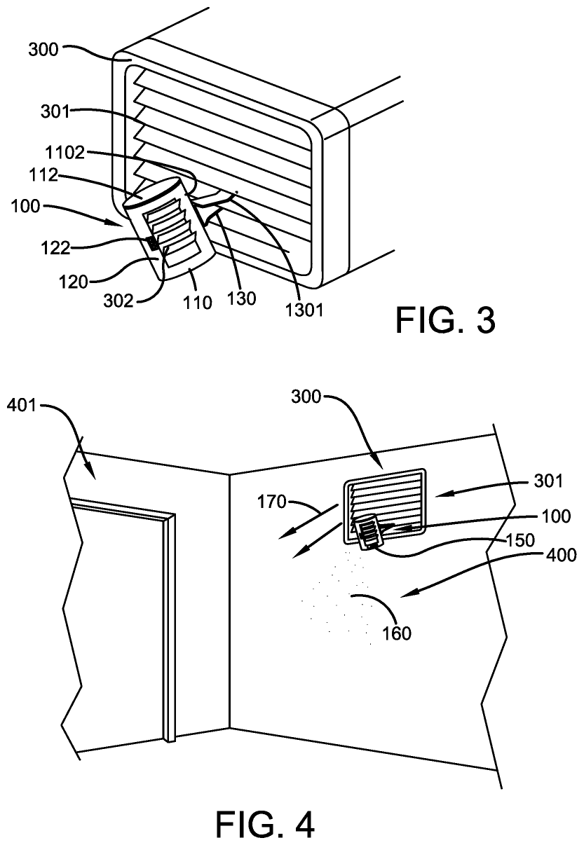

Notwithstanding, the air fresher device 100 may have a plurality of fragrance strength levels for the user to choose from (i.e., it's not limited to just the three referenced above). The mode selected by the user by sliding the button 1221 controls the opening and closing of the vents 302 on front surface 1100 of the housing, as best shown in FIGS. 3 and 4. For example, in the low strength mode 1223, the vents 302 are barely opened (i.e., less than 35%), thereby releasing only a small amount of the fragrant material 124 in the stream of forced air. Similarly, at medium strength mode 1224, the vents 302 are further opened but still less than 66% of full capacity, and therefore more fragrant material 124 is released from the air freshening device 100. At high strength mode 1225, all the vents 302 are 100% opened, and therefore the maximum possible amount of fragrant material 124 is released from the air freshening device 100 into the forced air stream. Further, the user may also choose to restrict the amount of fragrant material 124 released by device 100 by creating an angular displacement of the device 100 relative to the direction of the air flow. For example, positioning the device 100 at approximately 30 degrees relative to the direction of the air flow (as opposed to being directly in line with the air flow) is believed to reduce the flow rate (and accompanying amount of fragrant material released therein) by about one third.

The fragrance strength adjusting mechanism 122 is preferably attached to an outer body of the device 100, such as housing 110, to provide easy access to the user. However, the position of the fragrance strength adjusting mechanism 122 is not limited to the outer surface, and the strength adjusting mechanism 122 can be positioned at any position as per the user preferences, including hidden within housing 110 and wirelessly adjusted. More specifically, for controlling the device 100 remotely, the user may install a corresponding software application on a compatible smartphone device or any other electronic device, and may connect to the air freshening device 100 over a wireless network connection such as Wi-Fi, Bluetooth, NFC, RFID, or the like.

As previously stated, a user may control the amount of the fragrant materials 124 to be released from the device 100 by utilizing the fragrance strength adjusting mechanism 122 on the housing 110. The strength adjusting mechanism 122 can be in other forms, such as a touch button, a push button, a fragrance adjustment dial or the like. For example, in one embodiment having a plurality of adjustably sized openings in the housing 110, a user can control the amount of fragrant materials 124 released by turning a fragrance adjustment dial in a clockwise or counterclockwise motion, thereby modifying the size of the plurality of openings from large to small and vice versa.

The housing 110 structure may be made up of plastic, metal, or any other suitable material that provides a protective outer covering for the reservoir 120, and provides longevity to the air freshening device 100. Further, the housing 110 of the air freshening device 100 may be made up of surgical grade stainless steel, having anti-corrosion and/or hypoallergenic properties. The housing 110 may also be of any color, design, print, logo, trademark or the like 140 to enhance the aesthetic look of the air freshening device 100.

The reservoir 120 may be of a shape similar to the shape of the housing 110, such that the reservoir 120 fits inside the housing 110, or may take another shape which is easily removable from the housing and replaceable (e.g., a replaceable cartridge). More specifically, the reservoir 120 is removably coupled to the housing 110, and comprises a fragrant material 124. The fragrant material 124 may be in the form of a mesh, strips, a gel, sticks, solids, liquids, absorbent materials (e.g. sponges) and the like. The reservoir 120 can be accessed by opening the lid 112 and, once the reservoir 120 is accessed, the lid 112 can be closed.

In one embodiment of the present invention, the fragrant material 124 is a gel or other composition that is diffusible and exhibits no flow (or very slow flow) at room-temperature so that the gel or other material substantially maintains its shape during use. The fragrant material 124 may also have different scents such as lavender, rose, lemon grass, cinnamon, vanilla, pumpkin spice, combinations thereof or the like, and the fragrant material 124 can be selected as per the preferences of the users. Alternatively, a suitable fragrant material 124 may be made by mixing a desired fragrant oil in a liquid petroleum distillate diluent such as Isopar M, thereby producing the fragrant liquid 124.

As best shown in FIG. 2, the rear end 1102 of device 100 comprises a clamping mechanism or clip 130, and a plurality of repositionable openings or vents 202 for receiving forced air from an HVAC unit. The clip 130 is useful for removably attaching the device 100 to a forced air vent, and is also removably attached to the housing 110 at the back end 1102, which allows the air freshening device 100 to be easily fastened to any HVAC or other forced air emitting device. In a preferred embodiment, the clip 130 includes two extended flexible arms 1301 that engage into a vent or register grill of an air conditioner or heating system at home or a room unit, as best shown in FIGS. 3 and 4. The extended flexible arms 1301 are elongated strips connected together to provide the fastening mechanism. The flexible arms 1301 may also allow the device 100 to be disposed at a specific angular displacement relative to the vent in order to change the diffusion rate of the fragrant material 124, as explained more fully below.

Nonetheless, the extended flexible arms 1301 of the clip 130 can also be relatively short in length, or may be of any other shape as per the design requirements of the device 100. For purposes of illustration, but not limitation, attaching device 130 may be selected from any known devices in the art such as hook and loop fasteners, “S” hook fasteners, pins, barbs, clips, clamps, adhesives, tapes, magnetic fasteners, etc. The clip 130 enables the air freshening device 100 to be releasably attached in any of a horizontal, vertical or perpendicular position relative to the vent or register face, such that the openings in the device 100 are generally parallel to the direction of air flow, subject to the angular adjustment referenced herein. The clip 130 may be made of any suitable material, such as metal, plastic or the like. In a further embodiment of the present invention, the air freshening device 100 may be mounted to the vent, grill any other device, such as a vehicle air conditioner vent, table fans, or the like, using the clamping mechanism 130, which enables the device 100 to release the fragrant materials 124 into the surroundings through the force of the air received from the air flow device.

FIG. 2 illustrates a rear view of the air freshening device of the present invention in accordance with the disclosed architecture. The rear end 1102 of the housing 110 of the air freshening device 100 has a clamping clip 130 and vents 202. The clamping clip 130 includes extendible flexible arms 1301 used for mounting the air freshening device 100 on a vent or grill of any air flow or HVAC device in a room, vehicle, etc. The vents 202 may be opened on the rear surface 1102 of the housing 110, which enables the device 100 to receive the air blown from any air flow or HVAC type device, and further utilizes the force of the received air to release the fragrant or scented material 124 stored inside a reservoir 120 held inside the housing 110, into the surrounding area or room. As best shown on FIG. 3, additional vents 302 may be disposed on the front or outwardly facing surface of the housing 110, such that vents 202 and 302 may be disposed equally over the housing 110 to ensure even diffusing of the scented fragrant material 124. Alternatively, the vents 202 and 302 may be disposed only in the area of the reservoir 120 or supply of the scented material 124.

The clip 130 can be removed when the air freshening device 100 is not in use, and may be easily reattached to the rear surface 1102 of the housing 110 when the user needs to use the air freshening device 100. More specifically, the clip 130 can be removably attached to the housing 110 or the HVAC vent by means of a push lock mechanism, or the like. For purposes of illustration, but not limitation, attaching means may be selected from any means known in the art such as hook and loop fasteners, “S” hook fasteners, pins, barbs, clips, clamps, adhesives, tapes, magnetic fasteners, etc. In this manner, the clip 130 is easily replaceable in the event of loss or if the same becomes broken.

FIG. 3 illustrates a perspective view of one possible embodiment of an air freshening device 100 of the present invention in accordance with the disclosed architecture, wherein the air freshening device 100 is being attached to a forced air vent 302 of an HVAC unit such as an air conditioner 300. More specifically, a user (not shown) may hold the air freshening device 100 in a direction such that the rear end or surface 1102 of the housing 110 faces vent 301. A clip 130 or any other clamping mechanism can then be used to removably mount the air freshening device 100 to the vent 301 of the air flow device 300. More particularly, extended arms 1301 of the clip 130 are relatively flexible, and adjust to mount or clamp onto the vent 301. For optimum use, the device 100 may be positioned so that it is substantially parallel with the direction of air flow emanating from the vent, or angled from about 0 degrees up to about 45 degrees offset from the air flow direction, and more preferably between 0 degrees and 30 degrees. By using the clip 130 to position the device 100 in the angular arrangement, the amount of fragrant material 124 emanating from the device 100 and into the air flow stream can be further controlled.

In operation, the user turns on the HVAC device 300 so that it starts blowing a stream of air through the vents 301. As the air stream leaves the vent 301, it is forced to, in part, pass through the openings or vents 202 on the back 1102 of the housing 110. A reservoir 120 having fragrant material 124 receives the air from the HVAC unit through the vents 202 on the back 1102 of the housing 110 and infuses the same with the fragrant material 124 so that it is dispersed through the vents 302 present on the front surface of the housing 110 and into the entire room or other enclosed space. Further, the amount of fragrant material 124 released is easily controllable by the user via the angular placement of the device 100 relative to the vent 301 and/or by utilizing the small adjusting mechanism 122 as previously described on the housing 110 of the device 100.

FIG. 4 illustrates a perspective view of one potential embodiment of the air freshening device 100 attached to a vent 301 of an HVAC unit 300 in a room 410 in accordance with the disclosed architecture. More specifically, the air freshening device 100 is releasably attached to the vent 301 using a clamping mechanism or clip 130 as previously described and best shown in FIG. 2. The HVAC device 300 itself is mounted on a wall 400 of an enclosed space or a room 410, and is turned ON such that is emitting a stream of forced air through vent 301. As the stream of air is emitted from the HVAC device 300 it passes through the air freshening device 100 mounted on the vent 301. As described above, the forced air passing through the air freshening device 100 is diffused with the fragrant material 124 stored inside the reservoir of the device 100 and is released into the room 410. FIG. 4 also illustrates the angular positioning 150 of the device 100 relative to the vent 301, the particle size 160 of the fragrant material 124 being dispersed into the room 410, and the general direction of the air flow 170.

As previously mentioned, in an embodiment, a companion smartphone app is provided to regulate the operation of the freshener device 100. More specifically, the app connects to the device 100 through a Wi-Fi, Bluetooth, NFC or other wireless connection technology, where a small wireless connection chip (not shown) is present in the interior of the air freshener device 100. In this manner, a user can remotely customize the time, duration and scent level with functionality provided in the companion smartphone app.

In another embodiment, the air freshener device 100 of the present invention uses scented permeable material 124 protected through a shell or housing 110 that attaches to any forced air vent. More specifically, the fragrance is emitted whenever the forced air system is active and air passes through the scented permeable material. In one embodiment of the invention, the overall size of the freshening device 100 may be approximately four to six inches square for use in conjunction with a typically sized residential HVAC system. The device 100 is available with multiple fragrances 124, which may be phthalate-free, paraben free, and International Fragrance Association (IFRA) compliant.

In another embodiment, the air freshening device 100 of the present invention may use cold air diffusion technology, which works by using high amounts of vibration, such as ultrasonic vibrations to compress oils into a dry mist of nano particles of the size described above or 0.1 to 25 microns. The use of cold air diffusion technology ensures consistent and subtle scenting of a room or other enclosed space without high or low points in the scenting process.

In view of the foregoing, it can be seen that the air freshener device 100 of the present invention employs an air freshener releasing device 100 in combination with a forced air stream derived from a conventional forced air system normally installed in a house, office building or the like. More specifically, the air freshening device 100 is detachably connectable to an output vent, wherein the forced air emitted therefrom is forced past a reservoir of fragrant materials 124 to achieve distribution or a fragrance or scent throughout a room or other enclosed space. It should be noted that the air freshening device 100 of the present invention does not require the use of aerosols, harmful solvents, propellants, electricity, batteries, or child-endangering plug-in units. Further, the air freshening device 100 is comprised of environment friendly materials, and is both reusable and recyclable.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “air freshening device”, “freshener device”, and “fragrance diffuser” are interchangeable and refer to the air freshening device 100 of the present invention.

Notwithstanding the forgoing, the air freshening device 100 of the present invention can be of any suitable size and configuration as is known in the art without affecting the overall concept of the invention, provided that it accomplishes the above stated objectives. One of ordinary skill in the art will appreciate that the size, configuration and material of the air freshening device 100 as shown in the FIGS. are for illustrative purposes only, and that many other sizes of the air freshening device 100 are well within the scope of the present disclosure. Although the dimensions of the air freshening device 100 are important design parameters for user convenience, the air freshening device 100 may be of any size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A portable air freshening unit comprising;
a housing having a front and a rear surface, each of the front and rear surfaces having a plurality of vents;
an attachment device connected to the rear surface of the housing, the attachment device removably securing the housing to a vent of an air flow system; and
a reservoir containing a scented material.

2. The portable air freshening unit of claim 1, wherein the attachment device allows the rear surface of the housing to be angularly disposed from a face of the vent by up to 45 degrees.

3. The portable air freshening unit of claim 1, wherein the scented material is comprised of at least one of an anti-bacterial, an anti-viral, an anti-microbial and an anti-fungal solution.

4. The portable air freshening unit of claim 1, wherein the attachment device is a select one of a clip or a clamp.

5. The portable air freshening unit of claim 1, wherein the scented material is dispersed from the reservoir in a plurality of particles having a size of between 0.1 to 25 microns when exposed to an air stream being emitted from the vent of the air flow system.

6. The portable air freshening unit of claim 1, wherein the air flow system uses cold air diffusion.

7. The portable air freshening unit of claim 1, wherein the housing further comprises a fragrance adjusting mechanism to change a diffusing rate of the scented material from the reservoir.

8. The portable air freshening unit of claim 1, wherein the scented material is phthalate-free, paraben free, and International Fragrance Association compliant.

9. The portable air freshening unit of claim 7, wherein the diffusing rate may be modified by an angular displacement of the rear surface of the housing relative to the vent of the air flow system.

10. The portable air freshening unit of claim 7, wherein the fragrance adjusting mechanism has a low, a medium and a high setting.

11. The portable air freshening unit of claim 7, wherein the fragrance adjusting mechanism is remotely controlled by a smart device.

12. An air freshening system comprising:
a forced air flow system provided in a room and having at least one vent; and
an air freshening device comprising a vented housing, a supply of scented materials, and a clip for attaching the air freshening device to the at least one vent, wherein the supply of scented materials is phthalate-free, paraben free and International Fragrance Association compliant.

13. The air freshening system of claim 12, wherein the forced air flow system generates an air flow of between 400 and 1,000 CFM, and at least a portion of the air flow passes through the vented housing and is infused with a portion of the supply of scented materials.

14. The air freshening system of claim 13, wherein the air flow is infused with a plurality of particles from the supply of scented materials having a diameter of between 0.1 to 25 microns.

15. The air freshening system of claim 12, wherein the air freshening device comprises a fragrance adjusting mechanism to change a diffusing rate of the supply of scented materials, and further wherein the fragrance adjusting mechanism has a low, a medium and a high setting.

16. The air freshening system of claim 15, wherein the fragrance adjusting mechanism is activated by a smart device.

17. The air freshening system of claim 12, wherein the air freshening device uses cold air diffusion to disperse a plurality of particles from the supply of scented materials.

18. A scent diffusing device comprising:
a container attached to a vent of an air flow system, the container comprising a housing with a plurality of vents disposed on each of a front and a rear surface of the housing;
the container comprising a supply of scented material, the supply of scented material comprising at least one of an anti-bacterial, an anti-viral, an anti-microbial and an anti-fungal solution and being phthalate-free; and
the container further comprising a clip and a scent adjusting device to control a diffusing of the supply of scented material into a room from at least a low to a high setting, wherein the clip being able to provide an angular displacement of the container relative to a face of the vent of the air flow system.

19. The scent diffusing device of claim 18, wherein the angular displacement ranges from 0 degrees to 45 degrees.

20. The scent diffusing device of claim 18, wherein the scent adjusting device is controlled by a smart device.
